(19) United States
(12) Patent Application Publication
(10) Pub. No.: US 2018/0369714 A1
(43) Pub. Date: Dec. 27, 2018

(54) Apparatus and Methods for Optimizing Carbon Dioxide Utilization in Supercritical Extraction

(71) Applicant: Michael Coffin, Sacramento, CA (US)

(72) Inventor: Michael Coffin, Sacramento, CA (US)

(21) Appl. No.: 16/016,133

(22) Filed: Jun. 22, 2018

Related U.S. Application Data
(60) Provisional application No. 62/523,596, filed on Jun. 22, 2017.

(51) Int. Cl. B01D 11/02; A61K 31/352; C11B 9/02; F04B 41/06

(52) U.S. Cl. CPC B01D 11/0203; A61K 31/352; A61K 2236/37; F04B 41/06; C11B 9/025

(57) ABSTRACT

A process and apparatus for increasing the mass flow of supercritical CO2 per unit of time over an extraction bed for extracting essential material from organic material. An auxiliary pump feed is configured to receive an auxiliary process stream from a main process stream discharged from an extraction bed containing organic material. An auxiliary pump is connected to the auxiliary pump feed and is configured to create negative pressure in said auxiliary pump feed, thereby siphoning the auxiliary process stream from the main process stream and pushing it through an auxiliary pump discharge, where it is recirculated back to the extraction bed with the main process stream, whereby the mass of supercritical CO2 in the amplified process stream flowing over the organic material in the extraction bed is greater than the mass of supercritical CO2 in the main process stream per unit of time.

### CROSS-REFERENCE TO RELATED APPLICATIONS

The present application claims the benefit of U.S. Provisional Application No. 62/523,596 filed on Jun. 22, 2017, incorporated by reference herein and for which benefit of the priority date is hereby claimed.

### FEDERALLY SPONSORED RESEARCH

Not applicable.

### SEQUENCE LISTING OR PROGRAM

Not applicable.

### FIELD OF INVENTION

The present invention relates to the process and apparatus for optimization of CO2 utilization in the supercritical CO2 extraction of essential materials from organic materials.

### BACKGROUND OF THE INVENTION

An invention is needed that specifically addresses the problem of underutilized CO2 in a supercritical CO2 extraction process. In such processes, the supercritical CO2 solvent is not completely saturated when circulating through the extraction bed during extraction leaving excess solvent capacity in the supercritical fluid as it leaves the extraction bed. If the unsaturated solvent were to be able to recirculate, greater extraction efficiency could be achieved.

In a typical system for the extraction of essential oils from organic materials, CO2 is introduced into the system at high pressure. A system pump pushes the supercritical CO2 through a system pump discharge to an extraction bed, where the supercritical CO2 flows over the organic material in the extraction bed where it acts as a solvent, extracting essential oils from the organic material. The supercritical CO2 exits the extraction bed by a main process discharge, where it then enters a first separator. The pressure is reduced in the first separator causing a phase change in which the supercritical CO2 changes to a CO2 gas. The separator captures some of the essential oils from the CO2 gas, and the CO2 gas then exits the first separator via a first separator discharge and is pushed into a second separator where additional essential oils are captured. The gas then exits the second separator via the second separator discharge. Depending on the requirements of the extraction, additional separators may be used. In the case of two separators, the CO2 gas is pushed through the second separator discharge to a condenser, where the CO2 gas goes through a phase change back to supercritical CO2 and is pushed out of the condenser through the system pump feed. A system pump draws the supercritical CO2 through the system pump feed and pushes it out through the system pump discharge, where it repeats the foregoing process.

There presently exists the need to provide more efficient and effective utilization of CO2 in supercritical CO2 extraction processes by passing more mass of CO2 over the organic material in extraction bed per unit of time. The present invention overcomes these limitations and provides other related advantages.

### SUMMARY OF THE INVENTION

A carbon dioxide amplification process for supercritical extraction is provided in which the mass flow of supercritical CO2 over an extraction bed is amplified by providing an auxiliary recirculation pump apparatus. In a system for the extraction of essential oils from organic materials, CO2 is introduced into the system at high pressure. A system pump pushes the supercritical CO2 through a system pump discharge to an extraction bed, where the supercritical CO2 initial process stream flows over the organic material in the extraction bed where it acts as a solvent, extracting essential oils from the organic material. The supercritical CO2 exits the extraction bed as the main process stream by a main process discharge. The main process discharge connects the extraction bed to the first separator. In one embodiment of the present invention, the proximal end of the auxiliary pump feed is connected to the main process discharge at a location between the extraction bed and the first separator. The distal end of the auxiliary pump feed is in turn connected to an auxiliary pump. A proximal end of an auxiliary pump discharge is connected to the auxiliary pump, and the distal end of the auxiliary pump discharge is connected to the system pump discharge at a location between the system pump and the extraction bed.

When the auxiliary pump is activated, negative pressure is introduced to the auxiliary pump feed, thereby pulling supercritical CO2 from the main process stream flowing through the main process discharge. This supercritical CO2 is pulled through the auxiliary pump as the auxiliary process stream and is pushed through the auxiliary pump discharge to the system pump discharge. When the auxiliary process stream enters the system pump discharge, it combines with the supercritical CO2 flowing from the system pump to the extraction bed to form the amplified process stream.

The amplified process stream then flows over the extraction bed where it provides increased solvating capacity by providing more mass of supercritical CO2 per unit of time to the organic material. After the supercritical CO2 exits the extraction bed as the main process stream which flows to the first separator via the main process discharge, with an amount of auxiliary process stream being siphoned off at the auxiliary pump feed. The pressure is reduced in the first separator causing a phase change in which the supercritical CO2 changes to a CO2 gas. The separator captures some of the essential oils from the CO2 gas, and the CO2 gas then exits the first separator via a first separator discharge and is pushed into a second separator where additional essential oils are captured. The gas then exits the second separator via the second separator discharge. Depending on the requirements of the extraction, additional separators may be used. In the case of two separators, the CO2 gas is pushed through the second separator discharge to a condenser, where the CO2 gas goes through a phase change back to supercritical CO2 and is pushed out of the condenser and into the system pump feed. A system pump draws the supercritical CO2 through the system pump feed and pushes it out through the system pump discharge, where it then combines with the auxiliary feed stream as it enters the system pump discharge at the auxiliary pump discharge to form the amplified process stream, which then flows over the extraction bed where it provides increased solvating capacity by providing more mass of supercritical CO2 per unit of time to the organic material than the initial process stream or the main process stream.

In one embodiment of the present invention, the system pump discharges the main process stream into the system pump discharge with the density of CO2 at 1200 psi of 0.4 grams per cubic centimeter, at a volumetric flow rate of between 1 and 1.5 liters per minute. In one embodiment of the present invention, the system pump discharges the main process stream into the system pump discharge with the density of CO2 at 1800 psi of 0.7 grams per cubic centimeter, at a volumetric flow rate of between 1 and 1.5 liters per minute. Upon activation, the auxiliary pump will divert a fraction of the output of the main process stream out of the extraction bed into the auxiliary pump feed. The auxiliary pump discharge will provide approximately 100 psi differential pressure, which represents approximately four times the differential across the extraction bed, and increases the total solvent flow across the extractor bed. Flow out the system pump; volumetric flow rate is basically the same 1 to 1.5 liters per minute; but when your solvent has higher density, you will move more mass. Range of mass flow depending on that density. An auxiliary pump with a volumetric flow rate of 1 to 1.5 liters per minute can increase the mass flow across the bed by twice.

In one embodiment of the present invention, the pressure at the auxiliary pump can be changed, and pressure can be modulated with a variable frequency device. In one embodiment of the present invention, a metering restriction valve can be used to adjust the speed of the auxiliary pump.

Efficiencies in extracting essential materials from organic materials may also be achieved by inline monitoring of the chemical composition of the main process stream. Specifically, with regard to terpene and cannabinoid extraction, increased quantity and quality of the extracted essential materials may be obtained.

Optimal terpene extraction can be accomplished through manipulating the parameters on the extraction bed. This may accomplished through a broad series of tests changing the pressure and the temperature in the extraction bed and separator vessels. The qualitative and quantitative elements in the terpene extract are measured. When a promising combination is identified, the process is repeated with different organic material from different producers. Promising parameters are identified that achieve very high terpene levels with very low cannabinoid levels and very low viscosity. Different strains have different extraction profiles.

In one embodiment of the present invention, data is gathered from the producers so that factors that the influence the terroir may be determined. In one embodiment, soil samples are used as a reference point. In one embodiment, factors are traced through generations of material from seed to sale. In one embodiment, an IoT system is implemented in which sensors are implemented throughout the supply chain. In one embodiment, sensors are placed to measure variables impacting the quality of the cultivation, including soil hydration, weather conditions, and other factors. Sensors may also be placed on the plants themselves. Sensors may also be placed anywhere along the supply chain.

When manipulating the temperature and pressure parameters, some surprising results may be obtained. When manipulating the temperature parameter, some ranges will cause blocks of dry ice to be generated in the separator vessel. The system pump, the auxiliary pump, the mass flow rates, the selectivity of the extraction, orifice size between extraction bed and separators, and system temperatures all have an influence how the pressure in a separator is established. When the extraction bed is run below ideal operating pressures and poor results obtained, manipulating the operating parameters may improve the essential material extracted. Time is also a significant factor; as the terpenes are exhausted in the organic material, more waxes are rendered; consequently the rendering of cannabinoid and wax increases as a function of time. As a result, the present invention strips the terpenes first. In one embodiment, data is obtained from the organic material prior to running the batch to establish the time parameter. A terpene profile of the organic material is established with the amount of milligrams per gram of terpene in the organic material. In one embodiment of the present invention, the light terpenes are specifically identified, the parameters are determined for how many minutes per kilogram the extraction process is run.

In one embodiment, there is data for a specific batch. A full batch is run multiple times, and approximately six months of data is used to confirm the profile. The inference from terpene profile, and in view of the first seven light terpenes, being the most volatile, that come off , the milligrams per gram can be determined, and that number enables the calculation of the number of hours per gram or hours per kilogram runtime on the terpene extraction. After that, the system can be reconfigured for cannabinoid extraction. When running under the terpene extraction parameters, when the amount of terpenes has been exhausted, all that will be rendered thereafter is wax, and therefore it would be more advantageous at that point to cease the terpene extraction and move to cannabinoid extraction.

Logging of data from the extraction process can assist in determining the extraction parameters. Logged information includes the temperature and pressure of the extraction bed, temperature and pressure of the separator(s), flow rate of coolant, delta t (temperature) of the coolant flow across all of the components across the system. That data can be used to determine how much heat was gained or lost in each major component of the system. The amount of CO2 can be characterized in each component of the system, and more importantly, the CO2 density in the extraction bed, and the enthalpy of the CO2 as it enters a separator and the boiling curve it creates to figure out how much liquid CO2 remains at the bottom and how much boiling vapor is at the top.

Lengthy experimentation to determine the time parameter to terpene extraction may be obviated by inline monitoring of the main process stream during the terpene extraction process. In one embodiment of the present invention, a sensor or tap is used to determine the amount of terpenes left in the main process stream. Terpenes absorb ultraviolet light in the 200 nm range. Cannabinoids absorb light in the 220 nm range, but do not absorb light in the 200 nm range. The main process stream can be tested for terpene content in the 200 nm range using a dynamic sight glass with sapphire windows and subjecting the material to ultraviolet lasers emitting at 200 nm. When the absorption of the ultraviolet light starts dropping, that indicates the optimal time to cease terpene extraction and reconfigure the extractor from the terpene extraction parameters to the cannabinoid extraction parameters. In one embodiment, testing for terpenes can be accomplished using UHPLC (liquid chromatography).

Inline monitoring of the main process stream during the terpene extraction process has the advantage of not requiring extensive experimentation using trial and error method of manipulating parameters. Additionally, such experimentation only yields useful information for the particular strain test. The present invention advantageously does not require any upfront experimentation and can be used for any strain. It can be very difficult to know a priori the terpene profile of any particular material, so having the ability to determine the tipping point of terpene extraction before contamination with waxes occurs without having to analyze six months of aggregated data is a tremendous step in efficiency.

There are multiple methods of determining when a particular terpene extraction has been completed, thereby triggering the communication to the extractor to reconfigure the extraction parameters from terpene extraction mode to cannabinoid extraction mode. Each of the inline monitors discussed is placed between the extraction bed and the first separator, and depending on the method used, either sampled periodically or continuously.

Methods of identifying optimal terpene extraction include UV adsorption, Fourier transform infrared spectroscopy (FTIR), dynamic Raman spectroscopy, and rapid supercritical fluid chromatography. Dynamic inline measurement can be performed during both terpene extraction and cannabinoid extraction. Initially, the parameters are set for terpene extraction to obtain the lower molecular weight products of interest, then another set of parameters are configured to extract the higher molecular weight products of interest with different polarity, namely the cannabinoids. In one embodiment of the present invention, both are concurrently monitored, and when the terpenes have been extracted, the system is reconfigured for cannabinoid extraction. Additionally, when inline monitoring determines that all of the H2O has been extracted, the system can be reconfigured for different conditions.

Inline monitoring enables fine tuning of the extraction process. As all of the molecules of interest are built on isoprene units, and ultimately a cannabinoid is also a terpene (albeit a large one), the heavier terpenes tend to come out with the cannabinoids.

It should also be noted that these processes can be used in combination. For example, you can use UV detection to do a bulk characterization. A limit can be set on the UV detector, and when the limit is hit, the system can switch to chromatography to obtain a sample and get a more precise measurement. The first three methods can be used as detectors on a small inline chromatography system. If a separation is taken and all of the compounds are separated out, then as they come across the column one at a time, a positive ID can be obtained using the three techniques.

UV as an initial screening to alter the system as to when a spectroscopy unit should be activated to get specific measurements. In one embodiment of the present invention, the UV detector is configured with a trigger limit, and when the limit is hit it will alert the system to switch to chromatography to obtain a sample and get a more precise measurement. UV is advantageous in that it allows a quick broad stroke examination of terpenes, but it is not specific.

In one embodiment of the present invention, hard UV at 190 nm is used to detect the presence of most of the light terpenes. In one embodiment, a periodic sampling method is used where there is a valve that is opened, some material is taken out, and then some spectroscopy is performed on the sample.

In another embodiment, there is continuous monitoring of the main process stream as a function of time. Specifically designed equipment is used, including a sample cell that can take 2000 psi, for example a flow through sample cell with sapphire site glasses that allows the UV light through to the material.

Fourier transform infrared spectroscopy (FTIR) is a technique which is used to obtain an infrared spectrum of absorption or emission of a solid, liquid or gas. An FTIR spectrometer simultaneously collects high spectral resolution data over a wide spectral range. FTIR spectra can be used with focus on presence of particular functional groups

There exists in the art IR probes that can immersed in material, and that will perform spectroscopy on the main process stream as it passes through the inline monitoring component. This method takes a broad spectrum picture and runs a fast Fourier transform against it to get a mass of numbers, and turns that into a series of peaks. The frequency of the peaks reveals quite a bit about the chemical composition in the main process stream. This method shows functional groups such as OH group, carboxyl acid, carbon-hydrogen bond, carbon double bonds (enes and conjugated ene systems). There also exist mathematical techniques that allow for quantitation of specific molecules in the matrix.

FTIR and Raman spectroscopy will provide specific molecule characterization by discerning a discrete wavelength or discrete compound coming across a column. While chromatography inline would be a five minute process, FTIR and Raman spectroscopy are advantageously instantaneous

Raman spectroscopy can be used with a laser in the IR region for fingerprint identification. In this method, a laser is directed towards the material, and certain wavelengths of light comprising the incidental Raman shift are detected.

In one embodiment of the present invention, dynamic Raman spectroscopy is used on the main process stream. Every molecule has a signature Raman spectra. A laser beam directed at a molecule will reflect back at a wavelength specific to that molecule; every molecule has a certain signature. Performing Raman spectroscopy on a feed flow allows the constituent molecules to be sorted. You can look for a specific terpene rather than specific class of terpenes, because a general class of terpenes has to do with where it is absorbing at 200nm, and that has to do with the conjugated [ene] system which pairs the double bonds together. Raman spectroscopy allows specific signatures to pop out .

In one embodiment of the present invention, a laser beam is directed to the main process stream. There is an incidental energy that comes off that is called the Raman shift. In one embodiment, a UV laser beam with a certain wavelength is directed to the main process stream, causing some light to bounce off to the side. This light is detected and measured; it is usually measured at 90 degrees off the beam angle, the incidence angle of the beam, and certain wavelengths of light are detected providing a clear fingerprint of a whole host of compounds. For example, if a wavelength at 1205 nm is detected, that will map back to a specific compound. A broad spectrum of wavelengths can be scanned. In one embodiment of the present invention, that system would look for selective wavelength analysis, and check for increase or decease in certain compounds to determine when to change parameters. For example, if it is detected that pinene has been depleted, the system can switch parameters to then extract the cannabinoids.

Rapid supercritical fluid chromatography can be used with the existing pressure in the system to flow through the chromatography column with a UV or diode array detector. It is similar to liquid chromatography, but you push a tiny amount of compound inline with CO2 system. It is similar to the use of a liquid chromatography machine, but there is a shorter custom column, using the existing pressure of the system to push a tiny aliquot out which is squirted through a chromatography column, which will separate everything out depending on polarity and other chemical characteristics. The target compound is detected based on its retention time in the column. This method performs inline chromatography using the pressure and solvent already in the machine.

It should be noted that CO2 utilization in terpene extraction could be increased in by increasing solvent mass flow across an extraction bed without an increase in mass flow of the existing pump. The system includes plumbing modifications to the system and the addition of an auxiliary high static pressure, low differential pressure pump. A loop is installed which takes fluid from the output of the extraction bed, into the auxiliary pump, then is pumped back to the inlet of the extraction bed thereby increasing mass flow across extraction bed. The pump is be able to withstand the static pressure of the supercritical CO2 (up to 5,000 psi) as well as provide enough differential pressure to pump the material across the extraction bed.

It should be noted that CO2 utilization in cannabinoid extraction could be increased in by increasing solvent mass flow across an extraction bed without an increase in mass flow of the existing pump. The system includes plumbing modifications to the system and the addition of an auxiliary high static pressure, low differential pressure pump. A loop is installed which takes fluid from the output of the extraction bed, into the auxiliary pump, then is pumped back to the inlet of the extraction bed thereby increasing mass flow across extraction bed. The pump is able to withstand the static pressure of the supercritical CO2 (up to 5,000 psi) as well as provide enough differential pressure to pump the material across the extraction bed.

### BRIEF DESCRIPTION OF THE DRAWINGS

A complete understanding of the present invention may be obtained by reference to the accompanying drawings, when considered in conjunction with the subsequent, detailed description.

FIG. 1 is a diagram of a supercritical CO2 extraction system with an auxiliary pump system.

FIG. 2 is a diagram of a supercritical CO2 extraction system with an inline monitor.

FIG. 3 is a diagram of a supercritical CO2 extraction system with an auxiliary pump system and an inline monitor.

### DETAILED DESCRIPTION

Before the invention is described in further detail, it is to be understood that the invention is not limited to the particular embodiments described, as such may, of course, vary. It is also to be understood that the terminology used herein is for the purpose of describing particular embodiments only, and not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed with the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a”, “an”, and “the” include plural referents unless the context clearly dictates otherwise.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, if dates of publication are provided, they may be different from the actual publication dates and may need to be confirmed independently.

### Definitions

Main process stream is the initial stream of supercritical CO2 that is introduced into the system or that is cycled through the system and is discharged through the system pump before it is combined with the auxiliary stream in the system pump discharge before entering the extraction bed. The stream exiting the extraction bed is also referred to as the main process stream.

Auxiliary process stream is the portion of supercritical CO2 that is diverted or siphoned off from the main process stream as it flows through the main process discharge on its way to the first separator.

Amplified process stream is the combined main process stream and auxiliary process stream that is combined as the auxiliary process stream enters the main process discharge from the auxiliary pump discharge.

Auxiliary pump feed is the tube or piping which connects to the main process discharge at a location between the extraction bed and the first separator. The auxiliary pump feed connects at the other end to the auxiliary pump.

Auxiliary pump is a pump which sits between the auxiliary pump feed and the auxiliary pump discharge, and when activated, draws the auxiliary process stream from the main process discharge through the auxiliary pump feed and pushes it to the system pump discharge through the auxiliary pump discharge. In one embodiment of the present invention, the auxiliary pump can withstand static pressure of up to 5,000 psi.

Auxiliary pump discharge is the tube or piping which connects to the system pump discharge at a location between the system pump and the extraction bed. The auxiliary pump discharge connects at the other end to the auxiliary pump.

System pump is a pump which sits between the system pump feed and the system pump discharge, and when activated, draws the main process stream through the system, proximally pulling the main process stream from the condenser through the system pump feed and pushing it to the extraction bed through the system pump discharge.

System pump discharge is the tube or piping which connects the system pump to the extraction bed. System pump discharge also connects to the auxiliary pump discharge at a location between the system pump and the extraction bed.

Extraction bed is the vessel containing the organic material for extraction.

Main process discharge is the tube or piping which connects to the extraction bed to the first separator.

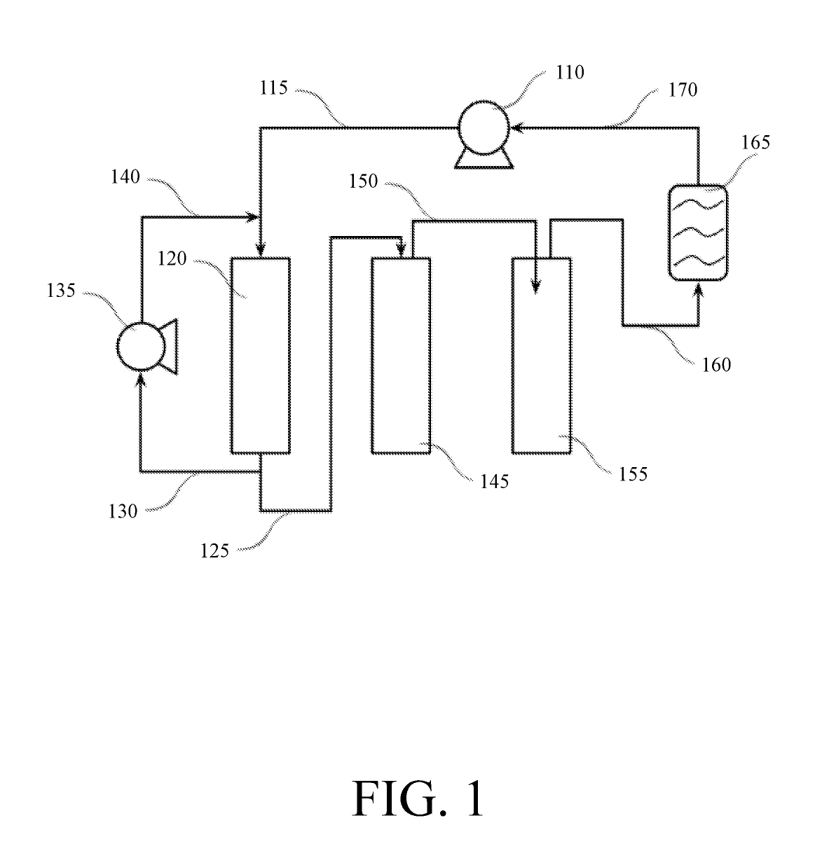

FIG. 1 shows a diagram of a supercritical CO2 extraction system which includes a carbon dioxide amplification process for supercritical extraction in which the mass flow of supercritical CO2 over organic material is amplified by providing an auxiliary recirculation pump apparatus. In a system for the extraction of essential oils from organic materials, CO2 is introduced into the system at high pressure. A system pump 110 pushes the supercritical CO2 through a system pump discharge 115 to an extraction bed 120, where the supercritical CO2 initial process stream flows over the organic material in the extraction bed 120 where it acts as a solvent, extracting essential oils from the organic material. The supercritical CO2 exits the extraction bed 120 as the main process stream by a main process discharge 125. The main process discharge 125 connects the extraction bed 120 to the first separator 145. In one embodiment of the present invention, the proximal end of an auxiliary pump feed 130 is connected to the main process discharge 125 at a location between the extraction bed 120 and the first separator 145. The distal end of the auxiliary pump feed 130 is in turn connected to an auxiliary pump 135. The proximal end of an auxiliary pump discharge 140 is connected to the auxiliary pump 135, and the distal end of the auxiliary pump discharge 140 is connected to the system pump discharge 115 at a location between the system pump 110 and the extraction bed 120.

Continuing with FIG. 1, when the auxiliary pump 135 is activated, negative pressure is introduced to the auxiliary pump feed 130, thereby pulling supercritical CO2 from the main process stream flowing through the main process discharge 125. This supercritical CO2 is pulled through the auxiliary pump 135 as the auxiliary process stream and is pushed through the auxiliary pump discharge 140 to the system pump discharge 115. In one embodiment of the present invention, the auxiliary pump 135 can withstand static pressure of up to 5,000 psi. When the auxiliary process stream enters the system pump discharge 115 from the auxiliary pump discharge 140, it combines with the supercritical CO2 flowing from the system pump 110 to the extraction bed 120 to form the amplified process stream.

Continuing further with FIG. 1, the amplified process stream then flows over the extraction bed 120 where it provides increased solvating capacity by providing more mass of supercritical CO2 per unit of time to the organic material. After the supercritical CO2 exits the extraction bed 120 as the main process stream which flows to the first separator 145 via the main process discharge 125, with an amount of auxiliary process stream being siphoned off at the auxiliary pump feed 130. The pressure is reduced in the first separator 145 causing a phase change in which the supercritical CO2 changes to a CO2 gas. The first separator 145 captures some of the essential oils from the CO2 gas, and the CO2 gas then exits the first separator 145 via a first separator discharge 150 and is pushed into a second separator 155 where additional essential oils are captured. The gas then exits the second separator 155 via the second separator discharge 160. In one embodiment of the present invention, depending on the requirements of the extraction, additional separators may be used. In the case of two separators, the CO2 gas is pushed through the second separator discharge 160 to a condenser 165, where the CO2 gas goes through a phase change back to supercritical CO2 and is pushed out of the condenser 160 through the system pump feed 170. The system pump 110 draws the supercritical CO2 through the system pump feed 170 and pushes it out through the system pump discharge 115, where it then combines with the auxiliary feed stream as it enters the system pump discharge 115 at the auxiliary pump discharge 140 to form the amplified process stream, which then flows over the extraction bed 120 where it provides increased solvating capacity by providing more mass of supercritical CO2 per unit of time to the organic material than the initial process stream or the main process stream.

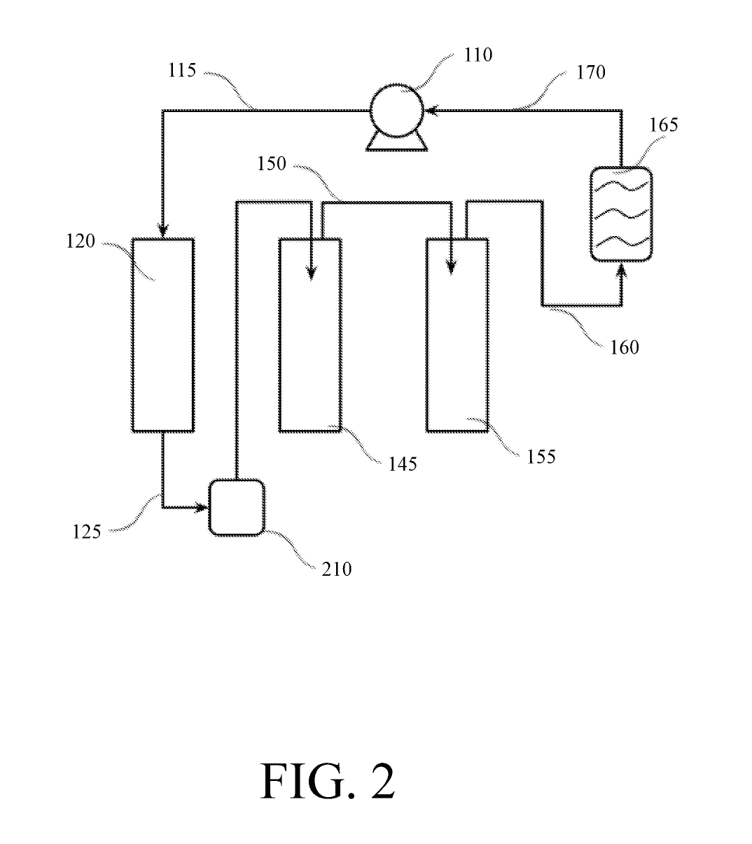

FIG. 2 shows a diagram of a supercritical CO2 extraction system which includes an inline monitoring process for supercritical extraction in which chemical composition of a main process stream can be optimized by monitoring for certain characteristics and changing extraction parameters based on the results provided by the inline monitor. In a system for the extraction of essential oils from organic materials, CO2 is introduced into the system at high pressure. A system pump 110 pushes the supercritical CO2 through a system pump discharge 115 to an extraction bed 120, where the supercritical CO2 initial process stream flows over the organic material in the extraction bed 120 where it acts as a solvent, extracting essential oils from the organic material. The supercritical CO2 exits the extraction bed 120 as the main process stream by a main process discharge 125. The main process discharge 125 connects the extraction bed 120 to the first separator 145. In one embodiment of the present invention, an inline monitor 210 is connected to the main process discharge 125 at a location between the extraction bed 120 and the first separator 145. The inline monitor 210 is configured to monitor the chemical composition of the main process stream as is passes through on its way to the first separator 145.

Continuing further with FIG. 2, after the supercritical CO2 exits the extraction bed 120 as the main process stream which flows to the first separator 145 via the main process discharge 125. The pressure is reduced in the first separator 145 causing a phase change in which the supercritical CO2 changes to a CO2 gas. The first separator 145 captures some of the essential oils from the CO2 gas, and the CO2 gas then exits the first separator 145 via a first separator discharge 150 and is pushed into a second separator 155 where additional essential oils are captured. The gas then exits the second separator 155 via the second separator discharge 160. In one embodiment of the present invention, depending on the requirements of the extraction, additional separators may be used. In the case of two separators, the CO2 gas is pushed through the second separator discharge 160 to a condenser 165, where the CO2 gas goes through a phase change back to supercritical CO2 and is pushed out of the condenser 160 through the system pump feed 170. The system pump 110 draws the supercritical CO2 through the system pump feed 170 and pushes it out through the system pump discharge 115, which then flows over the extraction bed 120.

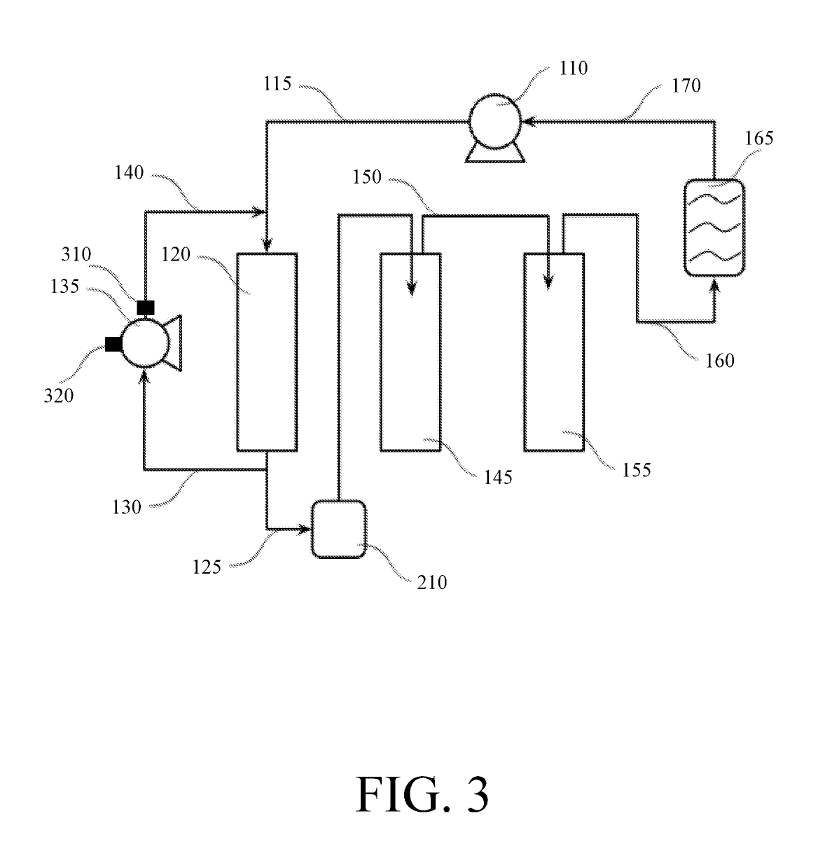

FIG. 3 shows a diagram of a supercritical CO2 extraction system which includes a carbon dioxide amplification process for supercritical extraction in which the mass flow of supercritical CO2 over organic material is amplified by providing an auxiliary recirculation pump apparatus an inline monitoring process for supercritical extraction in which chemical composition of a main process stream can be optimized by monitoring for certain characteristics and changing extraction parameters based on the results provided by the inline monitor. In a system for the extraction of essential oils from organic materials, CO2 is introduced into the system at high pressure. A system pump 110 pushes the supercritical CO2 through a system pump discharge 115 to an extraction bed 120, where the supercritical CO2 initial process stream flows over the organic material in the extraction bed 120 where it acts as a solvent, extracting essential oils from the organic material. The supercritical CO2 exits the extraction bed 120 as the main process stream by a main process discharge 125. The main process discharge 125 connects the extraction bed 120 to the first separator 145. In one embodiment of the present invention, the proximal end of an auxiliary pump feed 130 is connected to the main process discharge 125 at a location between the extraction bed 120 and the first separator 145 and an inline monitor 210 configured to monitor the chemical composition of the main process stream is connected to the main process discharge 125 at a location between the auxiliary pump feed 130 and the first separator 145. The distal end of the auxiliary pump feed 130 is in turn connected to an auxiliary pump 135. The proximal end of an auxiliary pump discharge 140 is connected to the auxiliary pump 135, and the distal end of the auxiliary pump discharge 140 is connected to the system pump discharge 115 at a location between the system pump 110 and the extraction bed 120. In an embodiment of the present invention, the inline monitor 210 is connected to the main process discharge 125 at a location between the extraction bed 120 and the first separator 145. The inline monitor 210 is configured to monitor the chemical composition of the main process stream as is passes through on its way to the first separator 145. In an embodiment of the present invention, data obtained by the inline monitor 210 is used to adjust the speed of the auxiliary pump 135 by sending an electronic signal to the metering restriction valve 310. In an embodiment of the present invention, data obtained by the inline monitor 210 is used to modulate the pressure generated by the auxiliary pump 135 by send an electronic signal to the variable frequency device 320.

Continuing with FIG. 3, when the auxiliary pump 135 is activated, negative pressure is introduced to the auxiliary pump feed 130, thereby pulling supercritical CO2 from the main process stream flowing through the main process discharge 125. This supercritical CO2 is pulled through the auxiliary pump 135 as the auxiliary process stream and is pushed through the auxiliary pump discharge 140 to the system pump discharge 115. When the auxiliary process stream enters the system pump discharge 115 from the auxiliary pump discharge 140, it combines with the supercritical CO2 flowing from the system pump 110 to the extraction bed 120 to form the amplified process stream.

Continuing further with FIG. 3, the amplified process stream then flows over the extraction bed 120 where it provides increased solvating capacity by providing more mass of supercritical CO2 per unit of time to the organic material. After the supercritical CO2 exits the extraction bed 120 as the main process stream which flows to the first separator 145 via the main process discharge 125, with an amount of auxiliary process stream being siphoned off at the auxiliary pump feed 130. The pressure is reduced in the first separator 145 causing a phase change in which the supercritical CO2 changes to a CO2 gas. The first separator 145 captures some of the essential oils from the CO2 gas, and the CO2 gas then exits the first separator 145 via a first separator discharge 150 and is pushed into a second separator 155 where additional essential oils are captured. The gas then exits the second separator 155 via the second separator discharge 160. In one embodiment of the present invention, depending on the requirements of the extraction, additional separators may be used. In the case of two separators, the CO2 gas is pushed through the second separator discharge 160 to a condenser 165, where the CO2 gas goes through a phase change back to supercritical CO2 and is pushed out of the condenser 160 through the system pump feed 170. The system pump 110 draws the supercritical CO2 through the system pump feed 170 and pushes it out through the system pump discharge 115, where it then combines with the auxiliary feed stream as it enters the system pump discharge 115 at the auxiliary pump discharge 140 to form the amplified process stream, which then flows over the extraction bed 120 where it provides increased solvating capacity by providing more mass of supercritical CO2 per unit of time to the organic material than the initial process stream or the main process stream.

It should be further understood that the examples and embodiments pertaining to the systems and methods disclosed herein are not meant to limit the possible implementations of the present technology. Further, although the subject matter has been described in a language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A carbon dioxide amplification process for supercritical extraction comprising:
providing a main process stream comprising supercritical CO2 into a system pump discharge;
pushing said initial process stream by activation of a system pump to an extraction bed containing organic material;
extracting essential material from said organic material in said extraction bed;
providing said main process stream to a main process discharge;
diverting an auxiliary process stream from said main process stream into an auxiliary pump feed by increasing negative pressure in said auxiliary pump feed by activating an auxiliary pump;
pushing said auxiliary process stream through an auxiliary pump discharge via said auxiliary pump;
providing said auxiliary process stream from said auxiliary pump discharge to said system pump discharge; and
providing an amplified process stream comprising said main process stream with said auxiliary process stream to said extraction bed.

2. The process of claim 1, wherein said auxiliary pump is capable of withstanding static pressure of 5,000 psi.

3. The process of claim 1, wherein said auxiliary pump stream provides a differential pressure in excess of 100 psi.

4. The process of claim 1, wherein said auxiliary pump provides a volumetric flow rate of between 1 and 1.5 liters per minute.

5. The process of claim 1, wherein said organic material comprises cannabis and said essential material comprises terpenes or cannabinoids.

6. The process of claim 1, further comprising modulating the pressure generated by said auxiliary pump with a variable frequency device. The process of claim 1, further comprising adjusting the flow rate of said auxiliary pump stream with a metering restriction valve.

8. The process of claim 6, further comprising monitoring the chemical composition of said main process stream with an inline monitor as it flows from said extraction bed to a separator.

9. The process of claim 8, wherein said variable frequency device modulates the said pressure generated by said auxiliary pump based on said chemical composition of said main process stream detected by said inline monitor.

10. An apparatus for increasing the mass flow of supercritical CO2 over an extraction bed, said apparatus comprising:
an auxiliary pump feed configured to receive an auxiliary process stream from a main process stream discharged from the proximal end of an extraction bed containing organic material;
an auxiliary pump, wherein said auxiliary pump is connected to said auxiliary pump feed and is configured to create negative pressure in said auxiliary pump feed, thereby siphoning said auxiliary process stream from said main process stream;
an auxiliary pump discharge, wherein said auxiliary pump discharge is proximally connected to said auxiliary pump and distally connected to a system pump discharge and configured to receive said auxiliary process stream from said auxiliary pump and discharge said auxiliary process stream into said system pump discharge, whereby said auxiliary process stream combines with a process stream to form an amplified process stream that is transported to the distal end of said extraction bed;
whereby the mass of supercritical CO2 in said amplified process stream flowing over organic material is said extraction bed is greater than the mass of supercritical CO2 in said main process stream per unit of time.

11. The apparatus of claim 10, wherein said auxiliary pump is capable of withstanding static pressure of 5,000 psi.

12. The apparatus of claim 10, wherein said auxiliary pump stream provides a differential pressure in excess of 100 psi.

13. The apparatus of claim 10, wherein said auxiliary pump provides a volumetric flow rate of between 1 and 1.5 liters per minute.

14. The apparatus of claim 10, wherein said organic material comprises cannabis.

15. The apparatus of claim 10, further comprising a variable frequency device, wherein said variable frequency devices is configured to modulate the pressure generated by said auxiliary pump.

16. The apparatus of claim 10, further comprising a metering restriction valve, wherein said metering restriction valve is configured to adjust the flow rate of said auxiliary pump stream from said auxiliary pump.

17. The apparatus of claim 10, further comprising an inline monitor connected to said main process discharge, wherein said inline monitor is configured to detect the chemical composition of said main process stream.
